(19) United States
(12) Patent Application Publication
(10) Pub. No.: US 2009/0280151 A1
(43) Pub. Date: Nov. 12, 2009

(54) FABRIC WITH ACTIVE PRINCIPLES AND RELEVANT RECHARGING METHOD OF THE ACTIVE PRINCIPLES

(75) Inventors: Morena Restani, Calderara Di Reno (IT); Lucio Lenzi, Vergato (IT)

Correspondence Address: Pearne & Gordon LLP, 1801 East 9th Street, Suite 1200, Cleveland, OH, 44114-3108, US

(73) Assignees: Morena RESTANI, Calderara Di Reno (Bologna) (IT); Lucio LENZI, Vergato (Bologna) (IT)

(21) Appl. No.: 12/440,210

(22) PCT Filed: Sep. 8, 2006
(86) PCT No.: PCT/IT06/00653

§ 371 Date: Jun. 23, 2009

(51) Int. Cl. A61K 9/00; A61P 3/10

(52) U.S. Cl. 424/404; 424/402

(57) ABSTRACT

The fabric consists of lycra fibres and microfibres and it has long the fibres a glycoprotein (9) and an exapeptide (10). The woof of this fabric and the active principles given to these two substances (9, 10) prevent and/or give the first cures to the diabetic foot.

### FIELD OF THE INVENTION

The present invention refers to a fabric with active principles and to a recharging method of said principles having the main aim to prevent and/or to give the first cures to the diabetic foot.

### BACKGROUND ART

Between the diabetes complications are known the problems to the feet caused to ulcers, accidental injuries, micro-traumas, wounds also light, callosity and infections. Are known the grave damages and the risks of grafting of important complications from light wounds in the diabetic patient. The persons with these pathologies have a reduced flux of blond in the inferior arts, in particular from the knee to the foot, owing to a stricture of the arteries. Moreover, the diabetes gives a progressive destruction of the nerves that arrive to the foot and to a skin degeneration, that becomes dry and inflexible, so easy to be subjected to wounds. The concomitance of these events determines infections often careless and minimal realized to the diabetic patient, this due also to a minor sensibility of the nervous terminations of the foot. The degenerative forms created to a light wound are, owing to the complications given to the metabolic, circulatory and neurological alterations, not to disregard as they easily evolve in serious pathologies, unfortunately favourite to long times for healing the wounds in the diabetic patient, with consequent increasing of risk of bacterial, viral and mycotic infections. The complications of a normal disregarded ulceration are one of the more frequent causes of admission to hospital for this pathology. The non-prevention and non-care of that known as diabetic foot determines the claudication and, in the grave cases, the amputation of the art. A consistent percentage of diabetic persons are subjected to ulcerations to the feet that prolong in the time can grow worse. To have an idea of the problem are cited the data of the Health World Organization where are counted in one hundred and twenty million the diabetic persons in year 1996 and it is estimated in three hundred million the number of diabetic persons in year 2025, where on an average the 15% will have ulcerations to the feet with complications. Always from the cited sources are known data that counted that for one hundred diabetic patients with amputations eighty-four have had as cause of the amputation an ulceration to a foot grows worse in time. Moreover, over the 50% of all the amputations of the lower limbs regards diabetic patients. Currently are not known local preventive applications to be constantly used to prevent, cure or reinforce the defences of the foot of the diabetic patient so to avoid the complications previous described. The sole forms of prevention in this delicate sector are given prevalently based onto mode of behaviour as the careful test of the food, the sanitary education for observation of hygienic rules, the using of correct shoes and the application of topical remedy at the appearing of the first ulcerations.

### SUMMARY OF THE INVENTION

The invention, instead, realizes a therapeutic remedy able to prevent the formation of ulcerations and to favourite the recovery of wounds eventually formed. The invention creates a fabric, for specific application in this pathology, impregnated with substances with synergetic action and gradual release, and a recharging method of said active substances when these have lost their efficacy in time. The recharging of the active principles to the fabric permits a reusing of the specific fabric and it permits to have a set of clothes with controlled release of the active substances.

### DETAILED DESCRIPTION OF THE INVENTION

The invention consists of a fabric 1 in lycra fibres and micro-fibres (known with the trade “meryl”), in variable percentages for application of this kind, that in actuation has the form of stocking 2, pantyhose 3, sock 4, winding bandage with closed border 5, bandage or plaster 6. This type of fabric, with new application for the specific pathology of the diabetic foot, has the characteristic to have fibres with minor or equal subtlety to ten micron. So the fibres of this fabric are of inferior diameter to that of the silk, that have a subtlety of the fibres of twelve micron, of the cotton with subtlety of the fibres of thirteen and half micron, of the cashmere with fibres diameter dimension of fifteen micron and of the wool with fibres diameter dimension of seventeen micron. The application of this characteristic of the invention is of big importance for the diabetic foot such as more the thread is thin minor are the damages to abrasion of the fabric onto the skin. So an extremely smooth fabric is obtained that no creates micro-abrasions onto the inelastic skin and dry of the diabetic patient. In other embodiment of the invention the fabric 1 is provided in the woof of thin silver yarns 7 with function anti-bacteric and bacteriostatic. To avoid grazes from sewing the invention, in the form of the stocking 2, pantyhose 3, sock 4, has a sewing with flat folding 8 in the anterior part of the stocking, imperceptible to the touch. The fabric 1, moreover, has elastic property (elasticity of 99-100%) so to not obstruct the venous flux, to not worsening the occlusive peripheral arterypatia and to favourite the local microcirculation. Unlike to the other synthetic fibres, moreover, it has capacity of umidity absorption, low rate of water retention and, differently to the natural fibres, quick time of drying. These characteristic have like resulting capacity to oppose the formation of fungus and to limit the bacteric growth. To the fabric 1 are then added active substances with application method to the fabric and recharging method of new conception. To the fabric 1 is bound a glycoprotein 9 (pseudoalteromonos ferment extract, known with the trade “antarcticine”) synthesized to a bacteria (pseudoalteromanas antartica NF3) of the species Gram-negative, with composition of the fat acids and to the proteinic outline typical of the alteromonas family. This substance during the cellular growth produces a glycoprotein exopolymer composed of the 76-86% of proteins and to 8-14% of sugar, in base of the used means. This active substance have had use, previous to the invention, like a cosmetic humectant and emollient ingredient and like a substance with crioprotective property. Said active principle, of new and original application in the treatment of the diabetic foot, has important property that opposed the dryness of the skin typical of the diabetic patient. It gives, infact, the coesion between the spinous and germinativum stratum favouring the madding and the folding of the interkeratociti liquids, so as estimated through the compression studies of the isotherms and of the multilayer of the Langmuir balance. Moreover, said substance stimulates the growth of the human skin fibroblast in the tests in vitro without the growth of the keratinociti, so acting onto the micro-ulcerations of the skin to their first turning up and onto all the cute surface, from the knee to the foot. Moreover, the active principle given to the cited substance increases the formation of elastin and of collagen type I and type IV hours in comparison, with a sample of skin not treated or treated with cream with the only placebo effect. This substance is inserted in the wet fabric in a quantity of at least 1 mg/ml. To the fabric 1 is then added, without order of insertion to the fabric but with modality of charge and recharge following described, an exapeptide 10 from the alpha chain (known with trade “serilesine”). This peptide increases the adhesion of the cells, favours the cellular proliferation and betters the microcirculation such as it favours the angiogenesis. This active substance is added to the wet fabric 1 in quantity at least of 0.05 mg/ml. Also this last substance is never previous used for the use in pathology such that of the diabetic foot. Both these substances are previous used in the cosmetic field. So they do not present toxicity problems and they are not to be considered drugs to be prescribed with prescription. In particular they do not have, such the made tests has certified, cytotoxicity on human fibroblasts, cytotoxicity on keratinocity, they have no given toxicity prove in the tests of hemolysis, in the tests of genotoxicity, i.e. in the Ames tests, and they are not irritating as valuing with the het-cam for the valuation of the potential ocular irritation. The used substances in the described concentrations do not create irritation to the skin, sensitization phenomenon, ocular irritation and oral and skin toxicity. These substances 9 and 10 are moreover bound to the fabric 1 essentially for coulomb bond, being they with prevalent positive charge whereas the fabric has fibres with negative charge for own constitution. The substances 9 and 10 are mixed into microcapsules 11 soluble in water. The fabric 1 is then washed in water at 50° C. Ending this, working the water is discharged and the fabric is wet with clean water to which is added a mordanting assistant 12, the nylofixan PM (known with trade “clariant”) that has the function to bound the substances contained in the microcapsules 11 to the aminic group of the fabric 1, by means of the polarization that favours the union between the fabric 1 and the substances 9 and 10 into the microcapsules.

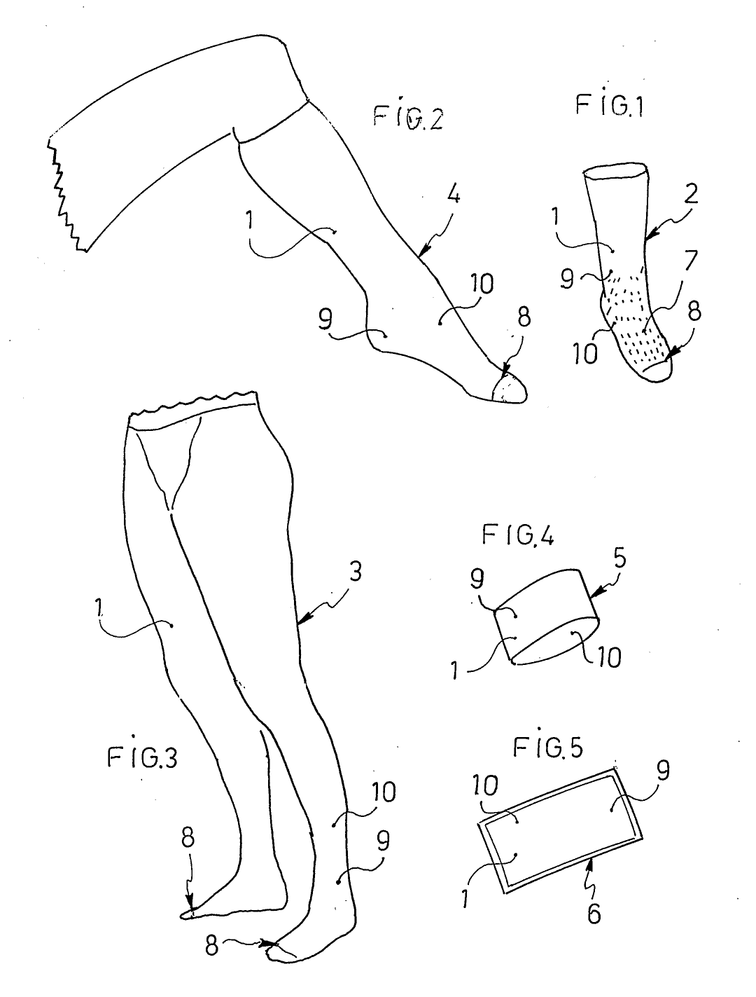

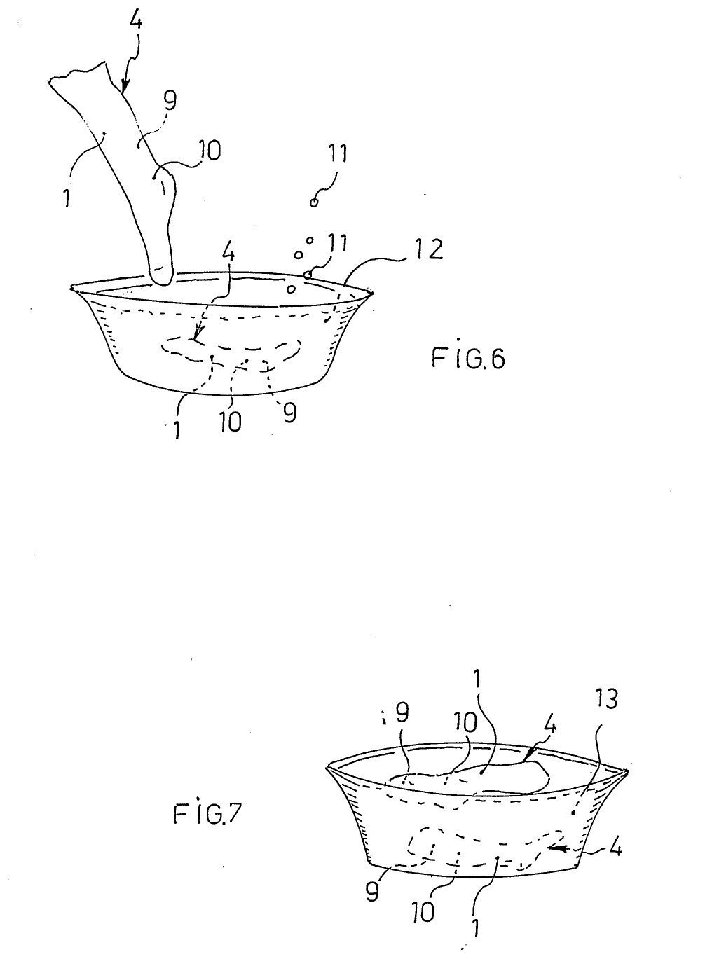

The fixer 12 is then added in proportion of 4% of the weight of the fabric 1 to treat. Then are introduced in the bath the microcapsules 11 in the dose previous described and in total quantity of the 6% of the weight of the fabric. After ten minutes is added a cationic product 13, i.e. softenol 70 (known with trade “lautex”) that contributes also it to bound the microcapsules 11 with the fabric 1. The “lautex” is added in percentage of 2% of the weight of the fabric. After ninety minutes the bath is let out and the fabric I is centrifuged and dry, ready to be used. In the version for the domestic use the microcapsules 11 and the fixers 12 and 13 to the fabric 1 are sold in pre-dosed packaging, to can use the stocking 2, the pantyhose 3, the sock 4, the winding bandage 5, the bandage or plaster 6. To actuate the recharging of the active substances 9 and 10 to the fabric 1 for domestic three grams of substances 9 and 10 in microcapsules 11 are brought and they are dissolved into 300-500 ml. of water, after to have washed the used fabric 1, at a temperature inferior to 40° C. The pantyhose, or the sock or the other forms of the fabric 1 are then left in soaking for 15-20 minutes. These products are then dry in air. After this treatment the fabric 1 have an uniform distribution of the active principles. With pre-determined times are provided rechargings, also conserving the active principles effectiveness till the third days of use. The invented product conserves the active principles also after three washings. However, it is suggested the daily washing and the recharging after three days from the first use with the method above described. The simple application of a cream with active principles does not reach the useful results for the diabetic foot pathology such as the results obtained with the invention such as the friction of the foot with the conventional pantyhose creates, during the walk, frictions that take off the cream instead of makes it to absorb to the skin of the foot and neither the occlusive bandages with cream and gauzes are not to be used if no with the patient in bed. The invention is so to be used in the daily without constraints and/or changing to the style of life of the patient. The invention in the application in the form of application also winding bandage 5, cicatrizant bandage or plaster 6 have application also remedy for the bedsores and/or light wounds. The cicatrizant action, the better of the micro-circulation and the effect on the derma determine a quick healing of these woods without collateral effects. The invention is illustrated in a merely indicative way in the drawings of sheets 1 and 2. In sheet 1 FIG. 1 is view of an example of production like a stocking. In the same table 1 the FIG. 2 is view of an application like a stock. FIG. 3 is view of the version like a pantyhose. FIG. 4 is view of the version of winding bandage with closed profile. FIG. 5 is view of the version of bandage or plaster. In sheet 2 are illustrated some sequences of the recharging phase of the active principles of the invention. In detail FIG. 6 is view of the immersion phase. FIG. 7 is view of the invention soaks in water with the active principles.

What we claim is:

1. Fabric with active principles and relevant recharging method of the active principles characterized in that a fabric (1) consists of lycra fibres and micro-fibres and it has distributed long the fibres a glycoprotein (9) and an exapeptide (10) and that the woof of said fabric and the active principles given to these two substances prevent and/or give the first cures to the diabetic foot.

2. Fabric with active principles and relevant recharging method according to claim 1, characterized in that the fabric (1) with the substances (9, 10) have the production form of a stocking (2), of a pantyhose (3), of a sock (4), of a winding bandage with closer border (5) of a bandage or plaster (6) so to realize a continuous and persistent contact with the skin of the diabetic patient.

3. Fabric with active principles and relevant recharging method according to claim 1, characterized in that the fabric (1) has in the woof thin silver yarns (7) for a anti-bacteric and bacteriostac function.

4. Fabric with active principles and relevant recharging method of the active principles according to claim 1, characterized in that the active principles (8, 9) are mixed into microcapsules (11) soluble in water.

5. Fabric with active principles and relevant recharging method according to claim 1, characterized in that to have the concentration of glycoprotein (9) to the wet fabric (1) in quantity of at least 1 mg/ml.

6. Fabric with active principles and relevant recharging method of the active principles according to claim 1, characterized in that to have the concentration of exapeptide (10) to the fabric in quantity at least of 0.05 mg/ml.

7. Fabric with active principles and relevant recharging method of the active principles according to claim 1, characterized in that the recharging or the first charging of the washed fabric is actuated bathing with clean water the fabric (1) to which is added a fixer (12), the nylofixan PM, in proportion of 4% of the weight of the fabric (1) to be treated, with the subsequent putting in water of soluble capsules (11) containing the active principles (8, 9) in total quantity equal to 6% of the weight of the fabric and, at the end, after ten minutes, is added the softenol 70 in percentage of 2% of the weight of the fabric and after ninety minutes the bath is discharge and the fabric (1) is centrifuged and drying, prompt to be used.

8. Fabric with active principles and relevant recharging method of the active principles according to claim 1, characterized in that to provide, for the recharging in domestic use, the use of three grams of the substances (9, 10) into the microcapsules (11) dissolve into 300-500 ml. of water at a temperature inferior to 40° C. and soaking of the fabric (1) for at least fifteen minutes.

9. Fabric with active principles and relevant recharging method of the active principles according to claim 1, characterized in that it is used like a remedy for the bedsores or for the light wounds.
